(19) United States
(12) Patent Application Publication
(10) Pub. No.: US 2012/0093949 A1
(43) Pub. Date: Apr. 19, 2012

(54) SYSTEMS AND METHODS FOR TREATING LICE

(76) Inventor: Ilene Steinberg, Wynnewood, PA (US)

(21) Appl. No.: 13/272,848

(22) Filed: Oct. 13, 2011

Related U.S. Application Data
(60) Provisional application No. 61/392,812, filed on Oct. 13, 2010.

(51) Int. Cl. A61K 36/889; A45D 24/00; A61K 36/534; A61P 17/04; A61K 36/63; A61K 36/61

(52) U.S. Cl. 424/727; 424/777; 424/742; 424/747; 132/219

(57) ABSTRACT

A method of treating lice includes the steps of: (a) blending a treatment mixture; (b) applying the treatment mixture to a scalp or hair; and (c) applying a first shampoo to the scalp or the hair. The treatment mixture used in this method may contain olive oil and dimethicone. The lice treatment mixture may further contain between about 2 and 6 drops of rosemary oil. In yet another embodiment the lice treatment mixture may still further contain at least one of the following ingredients: beeswax, vitamin E, citronella oil, tea tree oil, eucalyptus oil, mint oil, coconut oil, peppermint oil, and lavender oil.

### CROSS REFERENCE TO RELATED APPLICATIONS

This application claims priority from U.S. Provisional Patent Application No. 61/392,812 filed Oct. 13, 2010 which is incorporated herein by reference.

### FIELD OF INVENTION

This disclosure relates to systems and methods for treating lice. More particularly, this disclosure relates to systems and methods to eliminate any stage louse which can be applied to any level of lice infestation.

### BACKGROUND

Head lice treatment systems have been contemplated and studied for centuries. Known remedies for the removal of head lice include picking and combing lice and their nits out of the hair and scalp. Other treatments include using toxic powders, nit combs, and shaving. Olive oil as also been used to treat lice. Olive oil may also soothe an itchy dry scalp, but may not completely eliminate the head lice.

In contrast, some typical prior treatments, which use different formulations, are applied on the first day and then applied again only after seven days. Meanwhile, other prior treatments are applied once followed by ten days of monitoring and physical examination to confirm that the head lice have been completely eliminated. It has been discovered that these prior approaches are less effective in preventing reinfestation than the treatment steps illustrated by FIG. 3. Head lice have a life cycle of seven to ten days. An individual bug may lay six to ten eggs (nits) per day. The eggs hatch in seven to ten days. The nymphs (the four stages of a baby louse) mature in seven to ten days, which is when a generation of lice can start to lay up to 200 eggs per louse in a lifetime.

### SUMMARY

A system and method for treating live head lice is provided. An embodiment of the method comprises the steps of: (a) blending a treatment mixture; (b) applying said treatment mixture to a scalp or hair; and (c) applying a first shampoo to said scalp or said hair.

The treatment mixture used in this method may comprise olive oil and dimethicone. The ratio of olive oil to dimethicone may be approximately 1 to 1. In another embodiment, an effective amount of beeswax may be added to the treatment mixture.

In one embodiment, the step of applying shampoo to hair takes place in the absence of water. In addition, the method may further comprise the steps of: (d) removing the first shampoo from said scalp or said hair; (e) applying a second shampoo to said scalp or said hair; and (f) removing the second shampoo from said scalp or said hair.

The method may further comprises the steps of: (g) repeating all of the above steps (a)-(f) or a subset of steps (a)-(f).

In an additional embodiment, a lice treatment mixture comprises: olive oil and dimethicone. The lice treatment mixture contains a ratio of said olive oil to said dimethicone of approximately 1 to 1. The lice treatment mixture may further comprise between about 2 and 3 drops of rosemary oils. The lice treatment mixture may further comprise between about 4 and 5 drops of rosemary oils. The lice treatment mixture may further comprise between about 5 and 6 drops of rosemary oils. In yet another embodiment the lice treatment mixture may still further comprise at least one of the following ingredients: vitamin E, citronella oil, tea tree oil, eucalyptus oil, mint oil, coconut oil, peppermint oil, and lavender oil. Additionally, the lice treatment mixture may comprise more than one of the following in combination vitamin E, citronella oil, tea tree oil, eucalyptus oil, mint oil, coconut oil, peppermint oil, and lavender oil.

In yet another embodiment the lice treatment system includes a nit comb comprising: a grip, a first grip width and a second grip width where the ratio of the first grip width to the second grip width is approximately 1.2, and where the ratio of first grip width to the grip length is approximately 1.2; a plurality of tines, comprising a tapered first tine end and where the tines are inserted into a tine receiving portion of the grip at a second tine end where the second tine end is oppositely disposed from the first tine end; wherein a ratio of an interval to a tine diameter is approximately 1.2; wherein a ratio of the grip depth to the tine diameter is approximately 4; and wherein a ratio of the tine length to the tine diameter is approximately 40.

In yet another embodiment the lice treatment mixture includes olive oil, dimethicone, and an effective amount of beeswax. The ratio of olive oil to dimethicone may be approximately 1. In yet another embodiment the treatment mixture may include approximately 30-80% olive oil, approximately 30-80% Dimethicone, approximately 0-2% rosemary oils and approximately 0.5-10% beeswax. In yet another embodiment the lice treatment mixture may further comprise: between about 2 and 3 drops of rosemary oils. The lice treatment mixture may further comprise between about 4 and 5 drops of rosemary oils. The lice treatment mixture may further comprise between about 5 and 6 drops of rosemary oils. In yet another embodiment the lice treatment mixture may still further comprise at least one of the following ingredients: vitamin E, citronella oil, tea tree oil, eucalyptus oil, mint oil, coconut oil, peppermint oil, and lavender oil. Additionally, the lice treatment mixture may comprise more than one of the following in combination: vitamin E, citronella oil, tea tree oil, eucalyptus oil, mint oil, coconut oil, peppermint oil, and lavender oil.

### BRIEF DESCRIPTION OF THE DRAWINGS

FIGS. 1 and 2 illustrate the ingredients of an example treatment mixture.

FIG. 3 illustrates example steps for applying a treatment mixture.

FIG. 4 is a front view of one embodiment of the nit comb.

FIG. 5 is a perspective view of one embodiment of a nit comb.

FIG. 6 is a side view of the nit comb.

FIG. 7 is an enlarged view of part of a tine of the nit comb.

FIG. 8 is a top view of a portion of the nit comb.

### DETAILED DESCRIPTION OF THE PREFERRED EMBODIMENT

Although applying olive oil to the scalp may be effective in relieving the itchiness that may accompany lice infestation, it has been discovered that treating head lice only with olive oil and a nit comb does not completely eliminate the head lice. Moreover, it has also been discovered that applying a treatment according to the typical two-week treatment schedule does not completely eliminate the head lice.

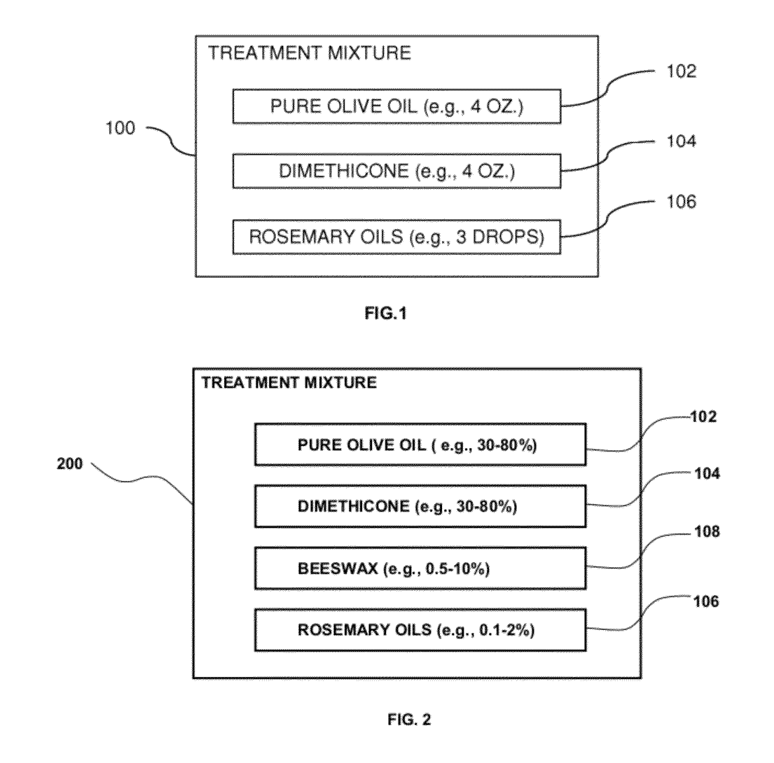

FIG. 1 illustrates a treatment mixture 100 that combines olive oil 102 with Dimethicone 104. Dimethicone can kill head lice within approximately ten seconds. As such, the treatment mixture 100 combines the lice-deterring, itch-relieving and moisturizing benefits of olive oil 102 with the ability of Dimethicone 104 to eliminate head lice. Further, it has been discovered that combining olive oil with Dimethicone allows for the preparation of an effective treatment to eliminate lice using smaller amounts of Dimethicone than were previously believed to be effective.

The treatment mixture 100 may also include additional ingredients including, but not limited to, Vitamin E, citronella oil, tea tree oil, eucalyptus oil, mint oil, coconut oil, peppermint oil, and lavender oil. Such ingredients act as a deterrent to lice infestation. As FIG. 1 illustrates, the treatment mixture 100 also includes rosemary oils 106. The rosemary oils 106 may act as a deterrent to lice infestation and provide the treatment mixture 100 with a more pleasant fragrance. Additionally, the rosemary oil prevents the olive oil from becoming rancid.

According to the example embodiment shown in FIG. 1, an eight ounce bottle of the treatment mixture 100 includes approximately four ounces of olive oil 102, approximately four ounces of Dimethicone 104, and approximately two or three drops of rosemary oil 106. Three drops of rosemary oil is approximately 0.0066 ounces (1 drop is approximately 1/480 of a U.S. fluid ounce). In another embodiment the lice treatment mixture may comprise between 2 and 6 drops of rosemary oils 106, including 3 drops, 4 drops or 5 drops of rosemary oils 106. It is understood, however, that the ingredients of the treatment mixture may be mixed in varying ratios. The treatment mixture 100 is stored at room temperature.

FIG. 2 illustrates an alternative embodiment of the treatment mixture 200 that further includes beeswax 108. Beeswax may increase the cohesiveness and thickness of the treatment mixture 100 an effective amount of beeswax 108 may be added. The beeswax 108 may also thicken the mixture so that it is less messy when applied to the hair or scalp and make the mixture more viscous.

According to the example embodiment shown in FIG. 2, an eight ounce bottle of the treatment mixture 200 includes approximately 19-80% olive oil 102, approximately 19-80% Dimethicone 104, approximately 0-2% rosemary oils 106 (including 0, 0.10, 0.20, 0.30, 0.40, 0.50, 0.60, 0.70, 0.80, 0.90, 1, 2%) and approximately 0.5-10% beeswax 108 (including 0.5, 0.6, 0.7, 0.8, 0.9, 1, 2, 3, 4, 5, 6, 7, 8, 9, 10%) to increase the cohesiveness of the treatment mixture. In an alternative embodiment the treatment mixture includes approximately 40-60% olive oil (including 40, 41, 42, 43, 44, 45, 46,47, 48, 49, 50, 51, 52, 53, 54, 55, 56, 57, 58, 59, 60%) and 30-40% Dimethicone (including 30, 31, 32, 33, 34, 35, 36, 37, 38, 39, 40%). The lice treatment mixture may further comprise between about 2 and 6 drops of rosemary oils 106, including 3 drops, 4 drops or 5 drops of rosemary oils 106. It is understood, however, that the ingredients of the treatment mixture may be mixed in varying ratios and percentages. The treatment mixture 200 is stored at room temperature.

Other ingredients that may be included in the treatment mixture 200 including, but not limited to, Vitamin E, citronella oil, tea tree oil, eucalyptus oil, mint oil, coconut oil, peppermint oil, and lavender oil. Like the rosemary oils, such ingredients act as a deterrent to lice infestation.

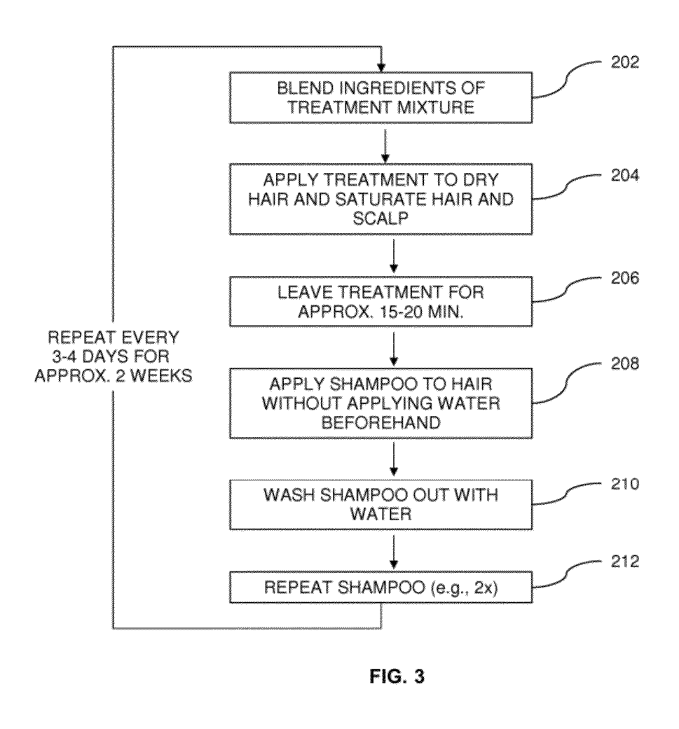

FIG. 3 illustrates example steps for applying the treatment mixture 100, 200. In step 202, the treatment mixture 100, 200 is blended, e.g., by vigorously shaking the bottle containing the treatment mixture. Due to their nature, the olive oil 102, Dimethicone 104, and the rosemary oils 106 may separate during storage. In step 204, the treatment mixture 100, 200 is applied to dry hair. In one embodiment the hair is wet during application. However, wet hair has the tendency to stick together and thus may prevent the treatment mixture 100, 200 from being applied properly. Moreover, the water in wet hair may keep the treatment mixture 100, 200 from saturating the hair effectively. Thus, wet hair may prevent the treatment mixture from being as effective as dry hair. The scalp and all of the hair are saturated with the treatment mixture 100, 200 in step 204. However, in an alternative embodiment only some of the hair is saturated with the treatment mixture 100, 200 in step 204. The eyes may be covered during the steps of FIG. 3 to prevent eye irritation. The treatment mixture 100, 200 is left in the hair for approximately fifteen to twenty minutes in step 206. In another embodiment the treatment mixture 100, 200 is left in the hair for approximately ten minutes in step 206. In yet another embodiment the treatment mixture 100, 200 is left in the hair for approximately five minutes in step 206. In another embodiment the treatment mixture 100, 200 is left in the hair for approximately three minutes in step 206. It is understood that the treatment mixture may remain in the hair for varying amounts of time including, 3, 4, 5, 6, 7, 8, 9, 10, 11, 12, 13, 14, 15, 16, 17, 18, 19 or 20 minutes.

In step 208, shampoo is applied to the hair and lathered without wetting the hair with water beforehand. In one embodiment of step 208 shampoo is applied to wet hair and lathered. Thus, in the alternative embodiment the hair is wet during application. However, wetting the hair beforehand may not be as effective because the shampoo may be affected by the possible separation of water and oil in the hair. Thus, the shampoo may not dissolve the oil as easily as it would without water. In step 210, the shampoo is washed out with water. The hair may be shampooed in step 212 one or two more times to achieve cleaner hair. Steps 202-212 are repeated every three to four days for a period of two weeks to prevent re-infestation.

The treatment mixture 100, 200 is more effective in preventing re-infestation when it is applied approximately every three to four days over two weeks. The treatment is sufficiently mild to allow daily applications if desired. However, for convenience, the treatment mixture 100, 200 can be applied every four days without affecting the efficacy of the treatment.

Two eight ounce bottles of the treatment mixture 100, including four ounces of the olive oil 102 and four ounces of the Dimethicone 104, may be provided in a treatment kit that generally provides four applications of the treatment mixture 100 over approximately two weeks. In general, four ounces of the treatment mixture 100 described above is sufficient to treat one head of hair having medium thickness. Although it is understood that other volumes of the olive oil 102 and Dimethicone 104 may be mixed in varying ratios, applying two ounces of the olive oil 102 and two ounces of the Dimethicone 104 every three to four days over two weeks is effective in providing relief and eliminating lice. Additionally, application of the treatment mixture 100, 200 every ten days after the initial two week treatment schedule minimizes the chances of another lice outbreak.

The treatment steps of FIG. 3 may eliminate the head lice as they hatch, before they reach maturity and are able to lay eggs. Additionally, the treatment steps of FIG. 3 may be combined with a housecleaning procedure. In known housecleaning procedures, anything made of fabric that touches the head of a person with a lice infestation should be laundered. Additionally, such an item should also be placed in a clothes dryer for at least 30 minutes at a temperature of at least 130° F. Hair accessories such as brushed, combs, barrettes, hair clasps, headbands, etc. should be boiled in water so that the hair accessories reach a temperature of at least 130° F.

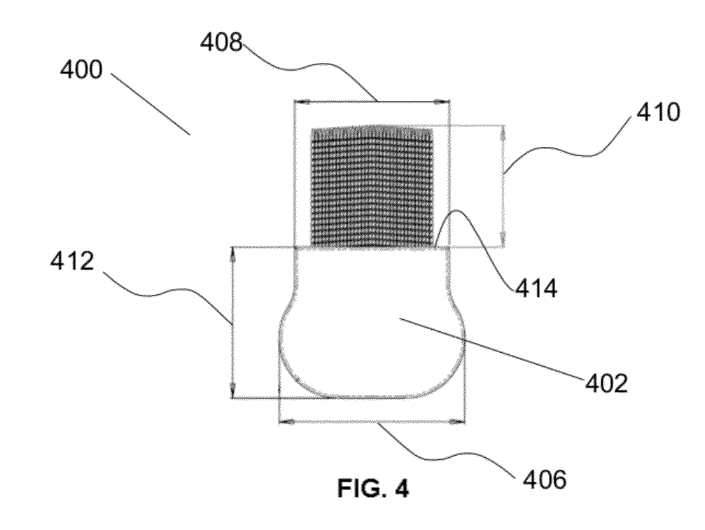

The application of the treatment mixture 100, 200 may be combined with the use of a nit comb, which physically removes the eggs (nits) from the hair. The olive oil 102 may also be helpful in loosening the nits from the hair. FIG. 4 illustrates a front view of one embodiment of a nit comb 400. It is to be understood however, that nit comb 400 is but one type of nit comb and other types of nit combs may be used with the above describe method. Metal combs with very fine teeth, such as stainless steel nit combs, are generally stronger and generally more effective than plastic nit combs. Removing eggs with a nit comb 400 may prevent the lice from returning if the treatment mixture 100, 200 fails to eliminate each of them.

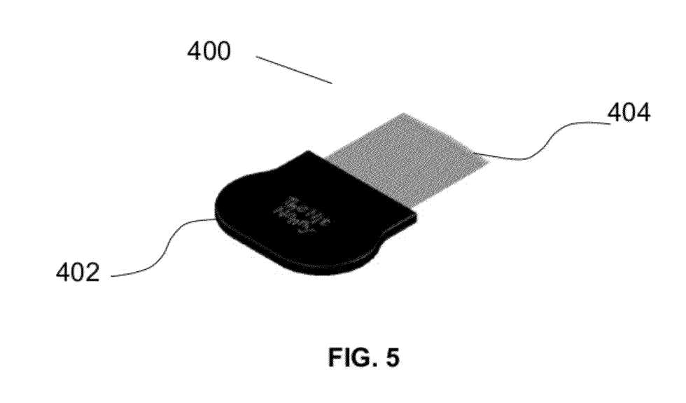

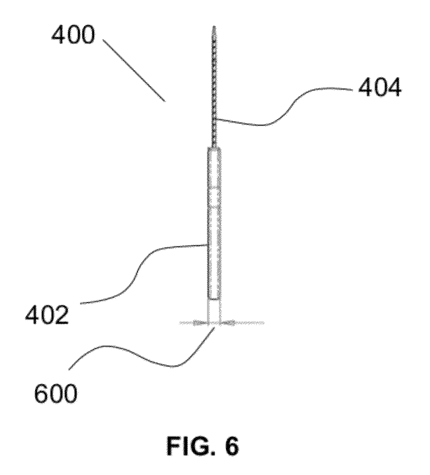

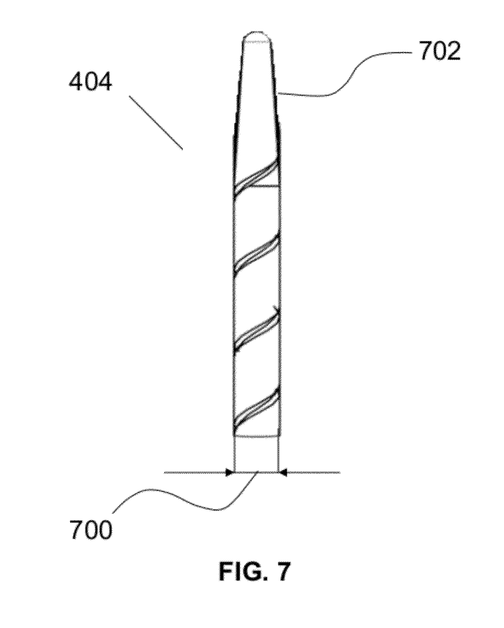

FIG. 4 is a front view of one embodiment of the nit comb. FIG. 5 is a perspective view of one embodiment of a nit comb. FIG. 6 is a side view of the nit comb. FIG. 7 is an enlarged view of part of a tine of the nit comb. The nit comb 400 includes a plurality of tines 404. In one embodiment the nit comb 400 includes approximately 34 tines 404, wherein each tine is tapered at a first tine end 702. The tines 404 are inserted into a tine receiving portion 414 of the grip. Additionally, the nit comb 400 also includes a grip 402. The grip 402 may be contoured such that the ratio of the first grip width 406 to the second grip width 408 is approximately 1.2. The ratio of first grip width 406 to the grip length 412 is approximately 1.2. The grip may be constructed of polypropylene. However, it is to be understood that any appropriate material may be used.

With reference to FIG. 6, the nit comb 400 has a grip depth 600. The nit comb has a tine diameter 700. The ratio of the grip depth 600 to the tine diameter 700 is approximately 4.

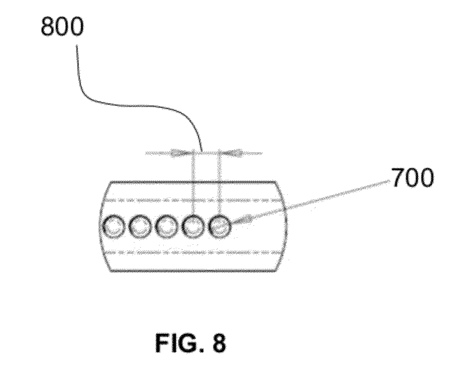

FIG. 8 is a top view of a portion of the nit comb. The distance between the center of a tine to the center of an adjacent tine is an interval 800. In one embodiment, the interval 800 may be less than 90 mm. In another embodiment, the interval 800 may be greater than 90 mm. A plurality of tines 404 may be present in the nit comb 400. Between 10 and 100 tines may be present. In one embodiment, the nit comb 400 has 34 tines. The ratio of the interval 800 to the tine diameter 700 is approximately 1.2. With reference now to FIGS. 4 and 7 the ratio of the tine length 410 to tine diameter 700 is approximately 40 to 1. The tines may be constructed of stainless steel, steel, or another sterilizable, rust proof metal.

### EXAMPLES

### Example 1

### In Vitro Test to Evaluate the Efficacy of Anti-Lice Products

The objective of this in vitro study was to analyze the efficacy of an anti-lice product treatment. The treatment solution was made from 4 oz. of dimethicone, 4 oz. olive oil and 3 drops of rosemary oil.

The subjects of the study were twenty children, over the age of 4 years. About 50 lice were collected in a jar, by combing the hairs of some of the 20 children.

Ten adult large lice were transferred to a lice chamber which consists of a cylindrical basket with a fine metal mesh bottom. The lice chamber was placed in a petri-dish, lined with white filter paper.

The petri-dish with the lice chamber were placed under a stereo-microscope with fiber optics illumination. The ten living lice were observed by their leg movements and constriction of the intestine.

The lice were then completely covered by the anti-lice product for 3 minutes. The lice chamber was then taken out of the petri-dish and the lice were washed three times for 30 seconds each with tap water. After washing, the lice chamber was placed over a fresh filter paper in a new petri-dish. With the stereo-microscope the living and dead (immobile) lice were counted, post washing, at 10 seconds, 10 min, 20 min, 30 min, 60 min, 2 hours and 4 hours sequentially.

TABLE 1.0
Results

| | 10 | 10 | 20 | 30 | 60 | 2 | 4 | 24 |
|---|---|---|---|---|---|---|---|---|
| | sec | min | min | min | min | hours | hours | hrs |
| Living lice | 0 | 0 | 0 | 0 | 0 | 0 | 0 | 0 |
| count | | | | | | | | |
| Dead | 10 | 10 | 10 | 10 | 10 | 10 | 10 | 10 |
| (immobile) | | | | | | | | |
| lice Count | | | | | | | | |

### Example 2

### Patch Test Results

Patch tests were conducted on 110 subjects from Aug. 20, 2011 until Aug. 24, 2011. The results show that of the 107 subjects who completed the testing, 107 showed no adverse response to the treatment mixture, which contained a mixture made from 4 oz. of dimethicone, 4 oz. olive oil and 3 drops of rosemary oil.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present disclosure has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the disclosure, in its broader aspects, is not limited to the specific details, the representative apparatus and method, and illustrative examples shown and described. Accordingly, departures may be made from such details without departing from the spirit or scope of the applicant's general inventive concept.

What is claimed is:

1. A method of treating lice comprising:
blending a treatment mixture;
applying said treatment mixture to a scalp or hair; and
applying a first shampoo to said scalp or said hair
wherein the treatment mixture comprises olive oil and dimethicone.

2. The method of claim 1 wherein a ratio of the olive oil to the dimethicone is approximately 1 to 1.

3. The method of claim 1 wherein the treatment mixture comprises:
19-80% olive oil;
19-80% Dimethicone;
0-2% rosemary oils; and
0.5-10% beeswax.

4. The method as defined by claim 1, wherein said step of applying said first shampoo to said scalp or said hair takes place in the absence of water.

5. The method as defined by claim 1, which further comprises:
physically removing nits from said scalp or said hair with a nit comb.

6. The method as defined by claim 1, which further comprises:
physically removing nits from said scalp or said hair with a nit comb wherein the nit comb comprises:
a grip having a tine receiving portion,
a plurality of tines, each tine having a tapered first tine end and a second end opposite the first end, where the second end of each tine is disposed in the tine receiving portion of the grip, and where each tine is separated by an interval where the interval measures a distance between a center of a tine to a center of an adjacent tine;
wherein a ratio of the interval to a tine diameter is approximately 1.2;
wherein a ratio of a grip depth to the tine diameter is approximately 4; and
wherein a ratio of a tine length to the tine diameter is approximately 40.

7. The method as defined by claim 1, which further comprises the steps of:
removing the first shampoo from said scalp or said hair;
applying a second shampoo to said scalp or said hair;
removing said second shampoo from said scalp or said hair.

8. The method as defined by claim 7, which further comprises:
blending a second treatment mixture;
applying the second treatment mixture to a scalp or hair;
applying a third shampoo to said scalp or said hair
removing the third shampoo from said scalp or said hair;
applying a fourth shampoo to said scalp or said hair; and
removing said fourth shampoo from said scalp or said hair.

9. The method as defined by claim 7, wherein the method is repeated at least every four days for a period of at least two weeks.

10. The method as defined by claim 7, wherein the method is repeated within ten days.

11. A lice treatment mixture comprising:
olive oil and dimethicone.

12. The lice treatment mixture of claim 11, wherein a ratio of said olive oil to said dimethicone is approximately 1 to 1.

13. The lice treatment mixture of claim 11 further comprising:
at least 2 drops of rosemary oil wherein 1 drop of said rosemary oil is approximately 1/480 of a U.S. fluid ounce.

14. The lice treatment mixture of claim 11 further comprising an ingredient selected from the group consisting of vitamin E, citronella oil, tea tree oil, eucalyptus oil, mint oil, coconut oil, peppermint oil, and lavender oil.

15. The lice treatment mixture of claim 11 further comprising beeswax.

16. The lice treatment mixture of claim 15 further comprising at least 2 drops of rosemary oil wherein 1 drop of said rosemary oil is approximately 1/480 of a U.S. fluid ounce.

17. The lice treatment mixture of claim 16 further comprising an ingredient selected from the group consisting of vitamin E, citronella oil, tea tree oil, eucalyptus oil, mint oil, coconut oil, peppermint oil, and lavender oil.

18. The lice treatment mixture of claim 11 wherein the treatment mixture comprises:
19-80% olive oil;
19-80% Dimethicone;
0-2% rosemary oils; and
0.5-10% beeswax.

19. A nit comb comprising:
a grip having a tine receiving portion,
a plurality of tines, each tine having a tapered first tine end and a second end opposite the first end, where the second end of each tine is disposed in the tine receiving portion of the grip, and where each tine is separated by an interval where the interval measures a distance between a center of a tine to a center of an adjacent tine;
wherein a ratio of the interval to a tine diameter is approximately 1.2;
wherein a ratio of a grip depth to the tine diameter is approximately 4; and
wherein a ratio of a tine length to the tine diameter is approximately 40.

20. The nit comb of claim 19 wherein the grip is constructed of polypropylene.
